(19) United States
(12) Patent Application Publication
(10) Pub. No.: US 2001/0019722 A1
(43) Pub. Date: Sep. 6, 2001

(54) Anti-acne chrono patch

(76) Inventors: Spiros Fotinos, Athens (GR); David P. O'Halloran, Milltown, NJ (US)

Correspondence Address: ALLEN BLOOM, C/O DECHERT, PRINCETON PIKE CORPORATION CENTER, P.O. BOX 5218, PRINCETON, NJ, 08543-5218, US

(21) Appl. No.: 09/757,416

(22) Filed: Jan. 9, 2001

Related U.S. Application Data
(63) Continuation-in-part of application No. 09/358,209, filed on Jul. 21, 1999.
Application No. 09/358,209 is a continuation of application No. 08/880,099, filed on Jun. 20, 1997, now Pat. No. 5,976,565.

(51) Int. Cl. A61K 31/185; A61K 31/21; A61K 9/70

(52) U.S. Cl. 424/448; 424/449; 514/553; 514/506

(57) ABSTRACT

Multi-layer, time release, anti-acne patches and multi-layer, anti-acne patches for the topical application of an anti-acne effective combination of active agents are provided along with a method for making the same. The multi-layer, anti-acne patches contain an anti-acne effective combination of at least two agents selected from the group of an antimicrobial agent, an antiseptic agent, an anti-irritant, a keratolytic agent, a hormone, a hormone agonist and a hormone antagonist. The individual layers of adhesive polymeric matrix material contained in the multi-layer, anti-acne patches provided, may contain identical or disparate combinations of agents constituting the anti-acne effective combination. The multi-layer, anti-acne patches may further contain enhancing agents which improve the efficacy of the anti-acne effective combination present therein.

This Application is a continuation-in-part of U.S. patent application Ser. No. 09/358,209 filed Jul. 21, 1999, which is a continuation of U.S. patent application Ser. No. 08/880,099 filed Jun. 20, 1997 (now U.S. Pat. No. 5,976,565).

The present invention relates to multi-layer, time release, anti-acne patches and multi-layer, anti-acne patches for the topical application of an anti-acne effective combination of active agents and a method of making the same.

Acne afflicts 90% of all teenagers. Acne also can afflict men and women in their twenties or thirties and in some cases may persist throughout adulthood. The process by which acne develops has been described in “New Approaches to Acne Treatment” by W. J. Cunliffe, ed. Martin Dunitz, London, 1989.

Acne vulgaris is a chronic disorder of the pilosebaceous follicles (apparatus) characterized by comedones (blackheads), papules, pustules, cysts, nodules, and often scars, that appear on the most visible areas of the skin particularly on the face, chest, back and occasionally neck, and upper arms.

The pilosebaceous apparatus is largely under the control of endogenous hormones (mainly androgens) which are present in unusually high concentrations in the blood during adolescence and puberty giving rise to an excessive production of sebum. The condition may worsen by a simultaneous increase in the rate of keratinization of the skin's horny layer (the stratum corneum). As the horny cells proliferate, they can form an occlusive plug or comedone which coupled with the increased production of the sebum, represents an ideal medium for the proliferation of the skin resident strains, such as the Gram positive anaerobic bacterium, Propionibacterium acnes.

Eventually, the plugged follicles rupture and allow the discharge of their contents causing local swelling and inflammation. The exposed follicles may darken from the deposition of pigment from damaged cells in the deeper layer of skin.

Acne is a multistage condition and in its most severe form can lead to hospitalization of the patient, extensive discomfort and long term scarring of the skin. There is a need for improved treatments for acne that will effectively prevent the condition from progressing to its most severe forms and that can be used by a majority of individuals without adverse effects.

### SUMMARY OF THE INVENTION

In a preferred embodiment of the present invention, multi-layer, time release, anti-acne patches are provided which include: a backing film, a release layer and at least two adhesive polymeric matrix layers. In a preferred aspect of this embodiment, the at least two adhesive polymeric matrix layers contain an anti-acne formulation uniformly distributed therein. The anti-acne formulation contains an anti-acne effective amount of at least two agents selected from the group of an antimicrobial agent, an antiseptic agent, an anti-irritant, a keratolytic agent, a hormone, a hormone agonist and a hormone antagonist.

In one aspect, the multi-layer, time release, anti-acne patches of the present invention preferably include an anti-acne formulation containing (a) a keratolytic agent, preferably selected from the group of salicylic acid and its biologically active esters, benzoyl peroxide, sulfur, retinoic acid and its biologically active esters, a fruit acid and an alpha hydroxy acid; most preferably salicylic acid; and (b) an anti-irritant, preferably selected from the group of α-bisabolol, farnesol, chamomile extract and glycyrrhetinic acid; most preferably α-bisabolol.

In another aspect, the multi-layer, time release, anti-acne patches of the present invention preferably include an anti-acne formulation containing (a) a keratolytic agent, preferably selected from the group consisting of salicylic acid and its biologically active esters, benzoyl peroxide, sulfur, retinoic acid and its biologically active esters, a fruit acid and an alpha hydroxy acid; most preferably salicylic acid; (b) an anti-irritant, preferably selected from the group of α-bisabolol, farnesol, chamomile extract and glycyrrhetinic acid; most preferably α-bisabolol; and (c) an antiseptic agent, preferably selected from the group of triclosan (Irgasan DP 300), phenoxy isopropanol, resorcinol, chlorhexidine, povidone and iodine; most preferably triclosan (Irgasan DP 300).

In yet another aspect, the multi-layer, time release, anti-acne patches of the present invention preferably include an anti-acne formulation containing (a) a keratolytic agent, preferably selected from the group of salicylic acid and its biologically active esters, benzoyl peroxide, sulfur, retinoic acid and its biologically active esters, a fruit acid and an alpha hydroxy acid; most preferably salicylic acid; (b) an anti-irritant, preferably selected from the group of α-bisabolol, farnesol, chamomile extract and glycyrrhetinic acid; most preferably α-bisabolol; and (c) an antimicrobial agent, preferably selected from the group of penicillins, cephalosporins, other beta-lactam compounds, aminoglycosides, tetracyclines, erythromycin, antifungal agents and combinations thereof; preferably erythromycin, tetracycline, clindamycin and cephalosporin.

In another preferred embodiment of the present invention, multi-layer, anti-acne patches are provided which include: a backing film, a release layer and at least two adhesive polymeric matrix layers. In a preferred aspect of this embodiment, the at least two adhesive polymeric matrix layers contain an anti-acne effective combination of at least two agents selected from the group of an antimicrobial agent, an antiseptic agent, an anti-irritant, a keratolytic agent, a hormone, a hormone agonist and a hormone antagonist; wherein the different adhesive polymeric matrix layers contain different components of the anti-acne effective combination.

In one aspect, the multi-layer, anti-acne patches of the present invention preferably include an anti-acne effective combination containing (a) a keratolytic agent, preferably selected from the group of salicylic acid and its biologically active esters, benzoyl peroxide, sulfur, retinoic acid and its biologically active esters, a fruit acid and an alpha hydroxy acid; most preferably salicylic acid; and (b) an anti-irritant, preferably selected from the group of α-bisabolol, farnesol, chamomile extract and glycyrrhetinic acid; most preferably α-bisabolol.

In another aspect, the multi-layer, anti-acne patches of the present invention preferably include an anti-acne effective combination containing (a) a keratolytic agent, preferably selected from the group of salicylic acid and its biologically active esters, benzoyl peroxide, sulfur, retinoic acid and its biologically active esters, a fruit acid and an alpha hydroxy acid; most preferably salicylic acid; (b) an anti-irritant, preferably selected from the group of α-bisabolol, farnesol, chamomile extract and glycyrrhetinic acid; most preferably α-bisabolol; and (c) an antiseptic agent, preferably selected from the group of triclosan (Irgasan DP 300), phenoxy isopropanol, resorcinol, chlorhexidine, povidone and iodine; most preferably triclosan (Irgasan DP 300).

In yet another aspect, the multi-layer, anti-acne patches of the present invention preferably include an anti-acne effective combination containing (a) a keratolytic agent, preferably selected from the group of salicylic acid and its biologically active esters, benzoyl peroxide, sulfur, retinoic acid and its biologically active esters, a fruit acid and an alpha hydroxy acid; most preferably salicylic acid; (b) an anti-irritant, preferably selected from the group of α-bisabolol, farnesol, chamomile extract and glycyrrhetinic acid; most preferably α-bisabolol; and (c) an antimicrobial agent, preferably selected from the group of penicillins, cephalosporins, other beta-lactam compounds, aminoglycosides, tetracyclines, erythromycin, antifungal agents and combinations thereof; preferably erythromycin, tetracycline, clindamycin and cephalosporin.

In yet another preferred embodiment of the present invention, multi-layer, anti-acne patches are provided which include: a backing film, a release layer, a first adhesive polymeric matrix layer and a second adhesive polymeric matrix layer; wherein the first adhesive polymeric matrix layer is interposed between the backing film and the second adhesive polymeric matrix layer. In a preferred aspect of this embodiment, the second adhesive polymeric matrix layer contains at least one enhancing agent. In another preferred aspect of this embodiment, the two adhesive polymeric matrix layers collectively contain an anti-acne effective combination of at least two agents selected from the group of an antimicrobial agent, an antiseptic agent, an anti-irritant, a keratolytic agent, a hormone, a hormone agonist and a hormone antagonist. In yet another preferred aspect of this embodiment, the different adhesive polymeric matrix layers contain different components of the anti-acne effective combination.

In one aspect, the multi-layer, anti-acne patches of the present invention preferably include an enhancing agent selected from the group of (a) a skin barrier disrupter, preferably selected from the group of sodium lauryl sulfate and low molecular weight (C₆-C₁₀) alkyl phenol ethoxylates; (b) a penetration enhancer, preferably selected from the group of polar-lipids, fatty acids, low molecular weight ethoxylated fatty alcohols, solubilizers and alpha hydroxy acids; (c) a skin conditioner, preferably selected from the group of glycerol and urea; and (d) an anti-irritant, preferably selected from the group of α-bisabolol, farnesol, chamomile extract and glycyrrhetinic acid; most preferably α-bisabolol.

In another aspect, the multi-layer, anti-acne patches of the present invention preferably include a skin barrier disrupter in the second adhesive polymeric matrix layer selected from the group of sodium lauryl sulfate and low molecular weight (C₆-C₁₀) alkyl phenol ethoxylates; and, salicylic acid or a biologically active ester of salicylic acid in the first adhesive polymeric matrix layer.

In yet another aspect, the multi-layer, anti-acne patches of the present invention preferably include a penetration enhancer in the second adhesive polymeric matrix layer selected from the group of polar lipids, fatty acids, low molecular weight ethoxylated fatty alcohols, solubilizers and alpha hydroxy acids; and, a keratolytic agent in the first adhesive polymeric matrix layer selected from the group of salicylic acid and its biologically active esters, retinol and the biologically active esters or salts of retinol.

In still another aspect, the multi-layer, anti-acne patches of the present invention preferably include a skin conditioner in the second adhesive polymeric matrix layer selected from the group of glycerol and urea; and, a keratolytic agent in the first adhesive polymeric matrix layer selected from the group of an alpha hydroxy acid, salicylic acid and its biologically active esters and sulfur.

In yet still another aspect, the multi-layer, anti-acne patches of the present invention preferably include an anti-irritant in the second adhesive polymeric matrix layer selected from the group of α-bisabolol, farnesol, chamomile extract and glycyrrhetinic acid; most preferably α-bisabolol; and, a keratolytic agent in the first adhesive polymeric matrix layer.

In still yet another aspect, the multi-layer, anti-acne patches of the present invention preferably include an anti-acne effective combination containing (a) a keratolytic agent, preferably selected from the group of salicylic acid and its biologically active esters, benzoyl peroxide, sulfur, retinoic acid and its biologically active esters, a fruit acid and an alpha hydroxy acid; most preferably salicylic acid; and (b) an anti-irritant, preferably selected from the group of α-bisabolol, farnesol, chamomile extract and glycyrrhetinic acid; most preferably α-bisabolol.

In even yet another aspect, the multi-layer, anti-acne patches of the present invention preferably include an anti-acne effective combination containing (a) a keratolytic agent, preferably selected from the group consisting of salicylic acid and its biologically active esters, benzoyl peroxide, sulfur, retinoic acid and its biologically active esters, a fruit acid and an alpha hydroxy acid; most preferably salicylic acid; (b) an anti-irritant, preferably selected from the group of α-bisabolol, farnesol, chamomile extract and glycyrrhetinic acid; most preferably α-bisabolol; and (c) an antiseptic agent, preferably selected from the group of triclosan (Irgasan DP 300), phenoxy isopropanol, resorcinol, chlorhexidine, povidone and iodine; most preferably triclosan (Irgasan DP 300).

In yet even another aspect, the multi-layer, anti-acne patches of the present invention preferably include an anti-acne effective combination containing (a) a keratolytic agent, preferably selected from the group consisting of salicylic acid and its biologically active esters, benzoyl peroxide, sulfur, retinoic acid and its biologically active esters, a fruit acid and an alpha hydroxy acid; most preferably salicylic acid; (b) an anti-irritant, preferably selected from the group of α-bisabolol, farnesol, chamomile extract and glycyrrhetinic acid; most preferably α-bisabolol; and (c) an antimicrobial agent, preferably selected from the group of penicillins, cephalosporins, other beta-lactam compounds, aminoglycosides, tetracyclines, erythromycin, antifungal agents and combinations thereof; preferably erythromycin, tetracycline, clindamycin and cephalosporin.

In still another preferred embodiment of the present invention, multi-layer, anti-acne patches are provided which include: a backing film, a release layer, a first adhesive polymeric matrix layer and a second adhesive polymeric matrix layer; wherein the first adhesive polymeric matrix layer is interposed between the backing film and the second adhesive polymeric matrix layer. In a preferred aspect of this embodiment, the first adhesive polymeric matrix layer contains urea and the two adhesive polymeric matrix layers collectively contain an anti-acne effective combination of at least two agents selected from the group of an antimicrobial agent, an antiseptic agent, an anti-irritant, a keratolytic agent, a hormone, a hormone agonist and a hormone antagonist.

In yet still another preferred embodiment, a method for making a multi-layer, anti-acne patches of the present invention having two adhesive polymeric matrix layers is provided, which includes: (a) casting a first adhesive polymeric matrix layer on a liner; (b) laminating the product of (a) on a backing film; (c) casting a second adhesive polymeric matrix layer on a release layer; (d) removing the liner from the product of (d).

### BRIEF DESCRIPTION OF THE DRAWINGS

There are shown in the drawings certain exemplary embodiments of the present invention as presently preferred. It should be understood that the present invention is not limited to the embodiments disclosed as examples, and is capable of variation within the spirit and scope of the appended claims.

In the drawings,

FIG. 1 is a depiction of a multi-layer, anti-acne device of the present invention comprising two adhesive polymeric matrix layers.

### DETAILED DESCRIPTION

The term “topically acceptable carriers”, as used herein, means substances substantially lacking toxicity for human tissues.

The term “topical application”, as used herein, means directly laying on outer skin.

The term “stable”, as used in the specification is defined as possessing a shelf-life that extends for more than several weeks.

The term “effective amount”, as used herein, means an amount sufficient to provide an anti-acne effect.

The present invention provides methods and devices for treating patients affected by acne, where the device has been optimized for minimizing adverse effects and for maximizing efficacy and is simple and comfortable to use. The topical treatment of acne and acneiform diseases disclosed herein utilizes a multi-layer, anti-acne patch to achieve local anti-acne effects that result from the suppression of the proliferation of horny cells and microbes involved in the pathogenicity of acne and reduction in associated inflammation. The multi-layer, anti-acne patches of the present invention have been designed to effectively deliver anti-acne agents to the stratum corneum (the outermost layer of epidermis, exposed to external environment) and subsequently to penetrate into the pilosebaceous unit (in the dermis) where the acneiform condition originates, while having very limited penetration into the systemic circulation.

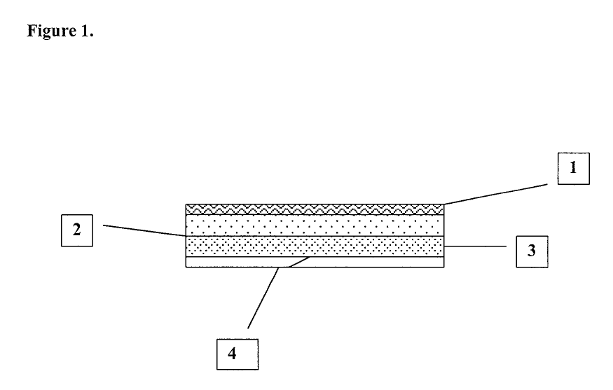

The preferred embodiments of the present invention will now be discussed with reference to FIG. 1 which generally depicts the preferred multi-layer configuration containing a backing film 1, a release layer 4 and two adhesive polymeric matrix layers 2 and 3. Notwithstanding, given the subject disclosure in connection with the following preferred embodiments, it will be readily apparent to a person skilled in the art that the multi-layer, anti-acne patches of the present invention may encompass devices containing more than two adhesive polymeric matrix layers.

Backing film materials suitable for use with the present invention include paper; cellophane; plastic films, for example polyethylene, polyester, polyurethane, polyvinyl chloride and polyamide; fabrics; metallic foils or composites thereof. The backing film material should be impermeable and non-reacting with the active agents contained in the multi-layer, anti-acne patches of the present invention. The backing film can be either transparent or opaque. In a preferred aspect, the backing film can be fleshton. The backing film preferably has a thickness ranging from 1 to 5 mils; more preferably 2 to 3.5 mils; most preferably 3 mils. Currently, the most preferred backing film materials include CoTran.TM.9720 (3M), Saranex.RTM.(Dow Chemicals) and Multilam fleshtoned polyester film 1009 (3M).

The release layer materials suitable for use with the present invention include materials impermeable to the substances dissolved in the adhesive polymeric matrix layers 2 and 3 and which are easily stripped off or released prior to use of the multi-layer, anti-acne patches of the present invention. Preferably, the release layer will be made from one of silicon, polyvinyl chloride, polyester, polyvinylidene chloride, polystyrene, polyethylene, paper or a combination thereof; more preferably an easy release silicon formulation, polystyrene film or siliconized polyester film. Currently, the most preferred release layer materials include a natural, high impact polystyrene film (grade code: 10106 or 15462) sold by REXAM Release and a siliconized polyester film sold by REXAM Release.

The multi-layer, anti-acne patches of the present invention include two or more adhesive polymeric matrix layers 2 and 3 which are interposed between the backing film 1 and the release layer 4. Materials suitable for use in the adhesive polymeric matrix layers of the multi-layer, anti-acne devices of the present invention include synthetic adhesives, for example acrylics, rubber, silicone, cellulosics, paper or other suitable materials that may have pressure sensitive adhesive properties and adhere to the skin directly or through a peripheral adhesive. Currently, the most preferred materials for use in the adhesive polymeric matrix layers include acrylic-based polymers (for example GELVA.RTM. series sold by Monsanto and the DUROTAK.RTM. series sold by National Starch), rubber-based polymers (for example DUROTAK.RTM. series sold by National Starch) and silicone-based polymers (for example BIO-PSA X7-4302 SILICONE PSA sold by Dow Coming). Alternatively, the adhesive polymeric matrix layers may contain paper materials, preferably Whatman filter paper, which is adhered onto the skin through a peripheral adhesive layer.

The adhesive polymeric matrix layers contained in the multi-layer, anti-acne patches of the present invention may include a variety of different agents composing an anti-acne formulation including antimicrobial agents, antiseptic agents, anti-irritants, keratolytic agents, hormones, hormone agonists and hormone antagonists. The anti-acne formulation collectively contained in the adhesive polymeric matrix layers may preferably further include solubilizers (for example glycerol, propylene glycol, polyalcohols, sorbitol and sorbitol derivatives; preferably sorbitan monooleate) and topically acceptable agents such as solvents, antioxidants and moisturizers.

Antimicrobial agents suitable for use in the multi-layer, anti-acne patches of the present invention include antimicrobial agents typically used for topical application, for example, penicillins, cephalosporins, other beta-lactam compounds, aminoglycosides, tetracyclines, erythromycin, antifungal agents and combinations thereof; preferably erythromycin, tetracycline, clindamycin and cephalosporin.

Antiseptic agents suitable for use in the multi-layer, anti-acne patches of the present invention include triclosan (Irgasan DP 300), phenoxy isopropanol, resorcinol, chlorhexidine, povidone and iodine; preferably triclosan (Irgsan DP 300).

Anti-irritants suitable for use in the multi-layer, anti-acne patches of the present invention include α-bisabolol, farnesol, chamomile extract and glycyrrhetinic acid; preferably α-bisabolol.

Keratolytic agents suitable for use in the multi-layer, anti-acne patches of the present invention include salicylic acid and its biologically active esters, benzoyl peroxide, sulfur, retinoic acid and its biologically active esters, any one of a number of fruit acids and alpha hydoxy acids; preferably salicylic acid.

The adhesive polymeric matrix layers contained in the multi-layer, anti-acne patches of the present invention may further include one or more enhancing agents. Enhancing agents suitable for use in the multi-layer, anti-acne patches of the present invention include skin barrier disrupters, penetration enhancers, skin conditioners and anti-irritants.

Skin barrier disrupters suitable for use in the multi-layer, anti-acne patches of the present invention include sodium lauryl sulfate and low molecular weight (C₆-C₁₀) alkyl phenol ethoxylates.

Penetration enhancers suitable for use in the multi-layer, anti-acne patches of the present invention include polar lipids, fatty acids, low molecular weight ethoxylated fatty alcohols, solubilizers and alpha hydroxy acids. Preferred polar lipids include plant polar lipids for example glycolipids, phospholipids and sphingolipids which may be extracted from wheat, rice, soya, millet and spinach. Preferred solubilizers include glycerol, propylene glycol, polyalcohols, sorbitol and sorbitol derivatives; most preferably glycerol. Preferred alpha hydroxy acids for use as penetration enhances in the multi-layer, anti-acne patches of the present invention include lactic acid and glycolic acid; most preferably lactic acid. Accordingly, it should be noted that any alpha hydroxy acids present in the multi-layer, anti-acne patches of the present invention may simultaneously serve two functions, namely they may serve as part of the anti-acne formulation and as a penetration enhancer.

Skin conditioners suitable for use in the multi-layer, anti-acne patches of the present invention include glycerol and urea.

Anti-irritants suitable for use as enhancing agents in the multi-layer, anti-acne patches of the present invention include α-bisabolol, farnesol, chamomile extract and glycyrrhetinic acid; preferably α-bisabolol. Accordingly, it should be noted that any anti-irritants present in the multi-layer, anti-acne patches of the present invention may simultaneously serve two functions, namely they may serve as part of the anti-acne formulation and as an enhancing agent.

In a preferred embodiment, the multi-layer, anti-acne patches of the present invention contain an anti-acne formulation composed of a combination of anti-acne agents including a keratolytic agent in combination with an anti-irritant, an antiseptic agent, an antimicrobial agent and/or other acne fighting compounds such as for example urea, allantoin, hydroxyquinoline compounds, for delivery via the patch directly to the area of skin to be treated. The presence of an anti-irritant counteracts the local irritation associated with the application of a keratolytic agent to the skin. The antiseptic limits the growth of organisms which cause the acne. Furthermore, the antimicrobial agent may enhance the overall anti-acne properties of the compositions in moderate to severe stages of the disease. The use of a solubilizer ensures that the active agents in the patch are in a form suited for diffusion from the patch to the skin.

In a preferred embodiment, the multi-layer, anti-acne patches of the present invention provide an anti-acne formulation containing, on a total weight of the carrier and the formulation basis:

(a) one or more keratolytic agent(s), each in an amount of 0.1 to 10.0% w/w, preferably of 0.1 to 2.0% w/w and more preferably of 0.6% w/w;

(b) one or more anti-irritant agent(s), each in an amount of 0.01 to 5.0% w/w, preferably of 0.01 to 3.0% w/w and more preferably of 1.0% w/w;

(c) one or more antiseptic agent(s), each in an amount of 0.05 to 2.0% w/w, preferably of 0.1 to 1.0% w/w and more preferably of 0.3% w/w; and,

(d) one or more solubilizer(s), each in an amount of 0.1 to 5.0% w/w, preferably of 1.0 to 3.0% w/w and more preferably of 2.0% w/w.

In a preferred embodiment, the present invention provides a multi-layer, time release, anti-acne patch as depicted in FIG. 1, including: a backing film 1, a release layer 4 and at least two adhesive polymeric matrix layers 2 and 3. In a preferred aspect of this embodiment, the at least two adhesive polymeric matrix layers 2 and 3 contain an anti-acne formulation uniformly distributed throughout both adhesive polymeric matrix layers. By distributing the same anti-acne formulation in equal proportions throughout two distinct adhesive polymeric matrix layers, it is believed that the delivery time for the active ingredients in the anti-acne formulation can be enhanced.

In another preferred embodiment, the present invention provides a multi-layer, anti-acne patch as depicted in FIG. 1, including: a backing film 1, a release layer 4 and at least two adhesive polymeric matrix layers 2 and 3. In a preferred aspect of this embodiment, the at least two adhesive polymeric matrix layers 2 and 3 contain different active agents. There are several reasons why it would be beneficial to have different active agents contained in the distinct adhesive polymeric matrix layers 2 and 3, for example, (a) certain combinations of active agents cannot be incorporated into a single adhesive polymeric matrix layer because (i) they exhibit different solubilities such that their incorporation into a single adhesive polymeric matrix layer would result in an unstable adhesive polymeric matrix layer, (ii) they would react with one another, for example salting out, if said agents were incorporated in a single adhesive polymeric matrix layer; and (b) the incorporation of certain combinations of actives in different layers may improve the efficacy of the patch, for example, the incorporation of a penetration enhancer in layer 3 may condition the skin at the target site, improving the activity of the active agent(s) present in layer 2.

In a preferred aspect of this embodiment, the present invention provides a multi-layer, anti-acne patch as depicted in FIG. 1, including: a backing film 1, a release layer 4 and at least two adhesive polymeric matrix layers 2 and 3. In this aspect, layers 2 and 3 would alternatively contain urea or salicylic acid acid and/or an alpha hydroxy acid. That is, in a given multi-layer, anti-acne patch of the present invention, layer 2 or 3 could alternatively contain salicylic acid acid and/or an alpha hydroxy acid while the other layer contains urea. One skilled in the art will recognize that (a) urea and (b) salicylic acid and/or an alpha hydroxy acid would be incompatible if contained in a single adhesive polymeric matrix layer because these agents would react together to form a less active ammonium salt. It would, however, be beneficial to incorporate such components within a given multi-layer, anti-acne patch. Specifically, it would be beneficial to follow up a treatment with an alpha hydroxy acid or salicylic acid by a treatment with urea, which is an excellent skin conditioner. Such a follow-up treatment with urea would operate to help restore the skin's natural moisturizing factor, return it to a natural pH and help to enhance the healing of any scars or lesions.

In another preferred aspect of this embodiment, the present invention provides a multi-layer, anti-acne patch as depicted in FIG. 1, including: a backing film 1, a release layer 4 and at least two adhesive polymeric matrix layers 2 and 3. In this aspect, layers 2 and 3 would alternatively contain a pH sensitive ester while the other layer contains some other active agent. That is, layers 2 or 3 could alternatively contain a pH sensitive esters such as retinol palmitate while the other layer contains an acidic or basic active combination. One skilled in the art will recognize that the stability of a combination of pH sensitive esters such as retinol palmitate with other actives could be improved if the pH sensitive esters are kept separate from the hostile environment created by certain other actives within a given adhesive polymeric matrix layer.

In yet another preferred aspect of this embodiment, the present invention provides a multi-layer, anti-acne patch as depicted in FIG. 1, including: a backing film 1, a release layer 4 and at least two adhesive polymeric matrix layers 2 and 3. In this aspect, layers 2 and 3 would alternatively contain an enhancing agent and an active agent. That is, in a given multi-layer, anti-acne patch of the present invention, layer 2 or 3 could alternatively contain an enhancing agent while the other layer contains an active agent or a combination of active agents, the activity of which is enhanced by the enhancing agent. One skilled in the art will recognize that the incorporation of certain enhancing agents in layer 3 can help prepare the skin for the delivery of certain active agents contained in layer 2. For example, one could incorporate a low molecular weight alpha hydroxy acid such as lactic acid or glycolic acid in layer 3 as an enhancing agent to enhance the activity of the less soluble salicylic acid active agent contained in layer 2. As another example, the use of skin barrier disrupters such as sodium lauryl sulfate or low molecular weight (C₆-C₁₀) alkyl phenol ethoxylates in layer 3 can function to reduce the barrier effect of the stratum cornium and thus facilitate the increased penetration of various active agents present in layer 2. Also, the incorporation in layer 3 of enhancers such as polar lipids, fatty acids, low molecular weight ethoxylated fatty alcohols, solubilizers and alpha hydroxy acids will increase the penetration of less polar active agents contained in layer 2 such as salicylic acid and its biologically active esters, retinol and its biologically active esters by increasing the intercellular lipids of the stratum comium. Further, the incorporation in layer 3 of skin conditioners that attract moisture to the skin such as glycerol or urea, would help to enhance the penetration from layer 2 of actives such as alpha hydroxy acids, salicylic acid and its more soluble salts, and sulfur. A preferred combination would include an alpha hydroxy acid in layer 3 and another keratolytic agent such as salicylic acid in layer 2.

In another embodiment, the present invention provides a method for making multi-layer, anti-acne patches. This method includes: (a) casting an adhesive polymeric matrix layer on a liner; (b) laminating the product of (a) on a backing film; (c) casting another adhesive polymeric matrix layer on another liner or a release layer; (d) removing the liner from the product of (a); (e) laminating the product of (c) to the product of (d); optionally (f) casting another adhesive polymeric matrix layer on another liner or release layer; (g) removing the liner from the product of (e); (h) laminating the product of (f) to the product of (g); (i) and continuing the process, adding one adhesive polymeric matrix layer at a time until the desired number of layers are obtained.

The present invention having been disclosed in connection with the foregoing embodiments, additional embodiments will now be apparent to persons skilled in the art. The present invention is not intended to be limited to the embodiments specifically mentioned, and accordingly reference should be made to the appended claims rather than the foregoing discussion, to assess the spirit and scope of the present invention in which exclusive rights are claimed.

We claim:

1. A multi-layer, time release, anti-acne patch, comprising: a backing film, a release layer and at least two adhesive polymeric matrix layers; wherein each polymeric matrix layer comprises an anti-acne formulation uniformly distributed therein, the formulation comprising an anti-acne effective amount of at least two agents selected from the group consisting of an antimicrobial agent, an antiseptic agent, an anti-irritant, a keratolytic agent, a hormone, a hormone agonist and a hormone antagonist.

2. The anti-acne patch of claim 1, wherein the anti-acne formulation comprises a keratolytic agent and an anti-irritant.

3. The anti-acne patch of claim 2, wherein the keratolytic agent is selected from the group consisting of salicylic acid, biologically active esters of salicylic acid, benzoyl peroxide, sulfur, retinoic acid, biologically active esters of retinoic acid, a fruit acid and an alpha hydroxy acid.

4. The anti-acne patch of claim 2, wherein the keratolytic agent is salicylic acid.

5. The anti-acne patch of claim 2, wherein the anti-irritant is selected from the group consisting of α-bisabolol, farnesol, chamomile extract and glycyrrhetinic acid.

6. The anti-acne patch of claim 2, wherein the anti-irritant is α-bisabolol.

7. The anti-acne patch of claim 2, wherein the keratolytic agent is salicylic acid and wherein the anti-irritant is α-bisabolol.

8. The anti-acne patch of claim 2, wherein the anti-acne formulation further comprises an antiseptic agent.

9. The anti-acne patch of claim 8, wherein the antiseptic is Irgasan DP 300, wherein the anti-irritant is α-bisabolol and wherein the keratolytic agent is salicylic acid.

10. The anti-acne patch of claim 2, wherein the anti-acne formulation further comprises an antimicrobial agent.

11. A multi-layer, anti-acne patch, comprising: a backing film, a release layer and at least two adhesive polymeric matrix layers; wherein the at least two adhesive polymeric matrix layers collectively comprise an anti-acne effective combination of at least two agents selected from the group consisting of an antimicrobial agent, an antiseptic agent, an anti-irritant, a keratolytic agent, a hormone, a hormone agonist and a hormone antagonist; and, wherein the different adhesive polymeric matrix layers comprise different components of the anti-acne effective combination.

12. The anti-acne patch of claim 11, wherein the anti-acne effective combination comprises a keratolytic agent and an anti-irritant.

13. The anti-acne patch of claim 12, wherein the keratolytic agent is selected from the group consisting of salicylic acid, biologically active esters of salicylic acid, benzoyl peroxide, sulfur, retinoic acid, biologically active esters of salicylic acid, a fruit acid and an alpha hydroxy acid.

14. The anti-acne patch of claim 12, wherein the keratolytic agent is salicylic acid.

15. The anti-acne patch of claim 12, wherein the anti-irritant is selected from the group consisting of α-bisabolol, farnesol, chamomile extract and glycyrrhetinic acid.

16. The anti-acne patch of claim 12, wherein the anti-irritant is α-bisabolol.

17. The anti-acne patch of claim 12, wherein the keratolytic agent is salicylic acid and the anti-irritant is α-bisabolol.

18. The anti-acne patch of claim 12, wherein the anti-acne effective combination further comprises an antiseptic agent.

19. The anti-acne patch of claim 18, wherein the antiseptic is Irgasan DP 300, wherein the anti-irritant is α-bisabolol and wherein the keratolytic agent is salicylic acid.

20. The anti-acne patch of claim 12, wherein the anti-acne formulation further comprises an antimicrobial agent.

21. A multi-layer, anti-acne patch, comprising: a backing film, a release layer, a first adhesive polymeric matrix layer and a second adhesive polymeric matrix layer; wherein the first adhesive polymeric matrix layer is interposed between the backing film and the second adhesive polymeric matrix layer; wherein the second adhesive polymeric matrix layer comprises at least one enhancing agent and wherein the two adhesive polymeric matrix layers collectively comprise an anti-acne effective combination of at least two agents selected from the group consisting of an antimicrobial agent, an antiseptic agent, an anti-irritant, a keratolytic agent, a hormone, a hormone agonist and a hormone antagonist.

22. The anti-acne patch of claim 21, wherein the at least one enhancing agent is selected from the group consisting of a skin barrier disrupter, a penetration enhancer, a skin conditioner and an anti-irritant.

23. The anti-acne patch of claim 22, wherein the at least one enhancing agent is selected from the group consisting of sodium lauryl sulfate and low molecular weight (C₆-C₁₀) alkyl phenol ethoxylates.

24. The anti-acne patch of claim 23, wherein the first adhesive polymeric matrix layer comprises salicylic acid.

25. The anti-acne patch of claim 22, wherein the at least one enhancing agent is selected from the group consisting of polar lipids, fatty acids, low molecular weight ethoxylated fatty alcohols, solubilizers and alpha hydroxy acids.

26. The anti-acne patch of claim 25, wherein the first adhesive polymeric matrix layer comprises a keratolytic agent selected from the group consisting of salicylic acid, biologically active esters and salts of salicylic acid, retinol, and the biologically active esters and salts of retinol.

27. The anti-acne patch of claim 22, wherein the at least one enhancing agent is selected from the group consisting of glycerol and urea.

28. The anti-acne patch of claim 27, wherein the first adhesive polymeric matrix layer comprises a keratolytic agent selected from the group consisting of an alpha hydroxy acid, salicylic acid and sulfur.

29. The anti-acne patch of claim 22, wherein the at least one enhancing agent is selected from the group consisting of an anti-irritant.

30. The anti-acne patch of claim 29, wherein the first adhesive polymeric matrix layer comprises a keratolytic agent.

31. The anti-acne patch of claim 21, wherein the anti-acne effective combination comprises a keratolytic agent and an anti-irritant.

32. The anti-acne patch of claim 31, wherein the keratolytic agent is selected from the group consisting of salicylic acid, benzoyl peroxide, sulfur, retinoic acid, a fruit acid and an alpha hydroxy acid and wherein the and wherein the anti-irritant is selected from the group consisting of α-bisabolol, farnesol, chamomile extract and glycyrrhetinic acid.

33. The anti-acne patch of claim 31, wherein the keratolytic agent is salicylic acid and wherein the anti-irritant is α-bisabolol.

34. The anti-acne patch of claim 31, wherein the anti-acne effective combination further comprises an antiseptic agent.

35. The anti-acne patch of claim 34, wherein the antiseptic is Irgasan DP 300, wherein the anti-irritant is α-bisabolol and wherein the keratolytic agent is salicylic acid.

36. The anti-acne patch of claim 31, wherein the anti-acne effective combination further comprises an antimicrobial agent.

37. A multi-layer, anti-acne patch, comprising: a backing film, a release layer, a first adhesive polymeric matrix layer and a second adhesive polymeric matrix layer; wherein the first adhesive polymeric matrix layer is interposed between the backing film and the second adhesive polymeric matrix layer; wherein the first adhesive polymeric matrix layer comprises urea and wherein the at least two adhesive polymeric matrix layers collectively comprise an anti-acne effective combination of at least two agents selected from the group consisting of an antimicrobial agent, an antiseptic agent, an anti-irritant, a keratolytic agent, a hormone, a hormone agonist and a hormone antagonist.

38. A method of manufacturing the multi-layer, time release, anti-acne patch of claim 21, comprising:
a) casting the first adhesive polymeric matrix layer on a liner;
b) laminating the product of (a) on the backing film;
c) casting the second adhesive polymeric matrix layer on the release layer;
d) removing the liner from the product of (b); and,
e) laminating the product of (c) to the product of (d).
